SEQUENCE LISTING (7 sequences)

SEQ ID NO: 1 - RNA, 21 nt, Artificial Sequence
  SiRNA sequence
aaccugcugaaggauggugac

SEQ ID NO: 2 - RNA, 21 nt, Artificial Sequence
  SiRNA sequence
aagaugacaucaccuggaccu

SEQ ID NO: 3 - DNA, 21 bp, Artificial Sequence
  Primer
aactggcaaaaggatggtgac

SEQ ID NO: 4 - DNA, 20 bp, Artificial Sequence
  primer
gccaggtgtcttagccagtc

SEQ ID NO: 5 - DNA, 20 bp, Artificial Sequence
  primer
gctccctcttgttgtggaag

SEQ ID NO: 6 - DNA, 20 bp, Artificial Sequence
  primer
atcgttttgctggtgtctcc

SEQ ID NO: 7 - DNA, 20 bp, Artificial Sequence
  primer
ctttgtggcaggtgtactgg

(19) United States
(12) Patent Application Publication
(10) Pub. No.: US 2006/0165665 A1
(43) Pub. Date: Jul. 27, 2006

(54) Immunomodulation using altered dendritic cells

(76) Inventors: Wei-Ping Min, London (CA); Thomas Ichim, Kitchener (CA); Jonathan Hill, London (CA)

Correspondence Address: SIM & MCBURNEY, 330 UNIVERSITY AVENUE, 6TH FLOOR, TORONTO, ON, M5G 1R7, CA

(21) Appl. No.: 10/517,275

(22) PCT Filed: Jun. 10, 2003
(86) PCT No.: PCT/CA03/00867

§ 371 Date: Aug. 1, 2005

(30) Foreign Application Priority Data
Jun. 10, 2002 (CA) 2388441

(51) Int. Cl. A61K 48/00; C12N 5/08

(52) U.S. Cl. 424/93.21; 435/372; 514/44

(57) ABSTRACT

The invention relates to altered immune cells and their use in methods and compositions to alter the immune system in a mammal. More specifically, the invention is directed to the alteration of gene expression in antigen presenting cells such as dendritic cells (DC) and their use in various methods and compositions to alter T cell activity for the treatment of a variety of immune disorders.

### FIELD OF THE INVENTION

The invention relates to altered immune cells and their use in methods to alter the immune system in a mammal. More specifically, the invention is directed to the alteration of gene expression in dendritic cells (DC) and their use in various methods to alter T cell activity for the treatment of a variety of immune disorders.

### BACKGROUND OF THE INVENTION

Throughout this application, various references are cited in parentheses to describe more fully the state of the art to which this invention pertains. Full bibliographic information for each citation is found at the end of the specification, immediately preceding the claims. The disclosure of these references are hereby incorporated by reference into the present disclosure.

Dendritic cells (DC) are the most potent antigen presenting cell (APC) endowed with the unique ability to stimulate and polarize naïve T cells to either Th1 or Th2 phenotypes (Maldonado-Lopez, R. et al., 2001. 13:275). DC also play a critical role in the maintenance of self tolerance by curtailing T cell responses directly or indirectly through the generation of T regulatory cells (Belz, G. T., et al., 2002. lmmunol Cell Biol 80:463; Mahnke, K., et al., 2002. Immunol Cell Biol 80:477; Min W. P. et al., J. Immunol. in press). The difference between DC subsets that stimulate and those that suppress immune responses seems to reside in the expression of co-stimulatory molecules and cytokines (Jonuleit, H., et al., 2001. Trends Immunol 22:394; Lu, L., et al., 2002. Transplantation 73:S19). The subset of DC called tolerogenic DC (Tol-DC) have a distinct phenotype, suppress activation of conventional T cells and activate T regulatory cells (Treg) in an antigen-specific manner (Chang, C. C. et al., 2002. Nat Immunol. Mar.; 3(3), 237-43; Gilliet M., et al.,2002. J. Exp. Med. March 18;195(6):695-704; Roncarlo, M. G. et al., 2001. J. Exp. Med. January 15;193(2):F5-9; Kawahata, K., et al., 2002. February 1; 168(3):1103-12.). Tol-DC possess reduced expression of the co-stimulatory molecules CD40, CD80 and CD86 and reduced ability to secrete T cell activating cytokines such as interleukin-12. Generally, expression of interleukin-12 (IL-12) seems to stimulate Th1 activation (O'Garra, A., et al., 1995. Res Immunol. 146:466), whereas production of IL-10 by DC stimulates Th2 activation (Liu, L., et al., 1998. Int Immunol 10:1017), and in some cases regulatory T cell generation (Akbari, O., et al., 2001. Nat Immunol 2:725; McGuirk, P., et al., 2002. J. Exp Med 195:221). Understanding this duality in function has led to DC based immunotherapies, which have been used to potentiate T cell responses (in the case of cancer vaccines) or diminish them (in autoimmune disorders and transplantation) (Pardoll, D. M. 1998. Cancer Vaccines. Nat Med 4:525; Morel, P. A. et al., 2001. Trends Immunol. 22:546; Prud'homme, G. J. 2000. J Gene Med 2:222).

Tolerogenic DC are generally in an immature state exemplified by suppressed expression of co-stimulatory molecules and IL-12. Various agents have been used to inhibit maturation of DC in order to promote tolerance. These agents are used to generate DC that express lower levels of co-stimulatory molecules. The proteosome inhibitor PSI (N-benzyloxycarbonyl-Ile-Glu(O-tert-butyl)-Ala-leucinal) blocks NF-KB activation and results in the in vitro production of tolerogenic DC (Yoshimura, S., et al., 2001. Eur J. Immunol. 2001 June;31(6):1883-93). N-acetylcysteine is an antioxidant which similarly blocks NF-KB activation and generates immature, tolerogenic dendritic cells (Verhasselt. V., et al., 1999. J. Immunol. March 1;162(5): 2569-74). Vitamin D3 also inhibits dendritic cell maturation and leads to production of tolerogenic dendritic cells (Piemonti L., et al., 2000. J. Immunol. May 1;164(9):4443-51). A disadvantage of using such agents is that there is no direct control of the resulting DC phenotype. Furthermore, DC exhibit plasticity in an in vivo environment which is disadvantageous for using DC directly in immunotherapy. Therefore the ability to generate DC with a specific phenotype and function would be advantageous.

Post-Transcriptional gene silencing is a mechanism that functions to inhibit viral replication in many eukaryotic organisms (Hannon, G. J. 2002. RNA Interference. Nature 418:244; Cogoni, C., et al., 2000. Curr Opin Genet Dev 10:638). This process is mediated by double stranded RNA (dsRNA) and can evoke many cellular reactions including the non-specific inhibition of protein synthesis seen in the interferon response of mammalian cells (Levy, D. E. et al., 2001. Cytokine Growth Factor Rev 12:143). It has recently been discovered that short sequences of RNA that are 21 nucleotides in length (known as small interfering RNA or siRNA) can bypass the broad suppression of the interferon response and can lead to the specific degradation of cognate mRNA (Elbashir, S. M., et al., 2001. Nature 411:494; Moss, E. G. 2001. Curr Biol 11 :R772). This process, known as RNA interference (RNAi), is specific as a single substitution in the 21 nucleotide sequence can abrogate its effects, and is extremely efficient, since the siRNA is incorporated into an enzymatic complex that conducts multiple rounds of target mRNA degradation (Tuschl, T. 2002. Nat Biotechnol 20:446). As such, RNAi provides a useful tool for inhibiting endogenous gene expression, and could provide a means to effectively modulate immune responses. Various methods of RNAi have been described for the altering gene expression in plant cells, drosophila and human melanoma cells as is described for example in U.S. Patent Application No. 2002/0162126A1, PCT/US01/10188, PCT/EP01/13968 and U.S. Patent Application No. 2002/0173478A1.

In general, RNA interference has been found to be unpredictable with low efficiency when used in vertebrate species (Fjose et al., Biotechnol. Annu. Rev. 7:31-57, 2001). Methods of RNA interference have not been previously contemplated for use in the transformation of immune cells and in particular the transformation of antigen presenting cells (APC) such as dendritic cells (DC) to produce a desired stable phenotype that can be further used in vitro, ex vivo and/or in vivo methods for the modulation of immune responses via the inhibition or stimulation of T cell activity. Furthermore, immune cells specifically designed to silence and thus suppress the expression of specific endogenous genes to affect T cell functioning have not been previously contemplated, nor contemplated for use in methods of treating immune disorders.

### SUMMARY OF THE INVENTION

The present invention provides immune cells that exhibit a targeted gene-specific knockout phenotype that can be used therapeutically to modulate immune responses in a mammal. More specifically, the present invention provides altered DC that do not express one or more genes encoding a molecule involved in DC activity, and as such, suppress or stimulate immune system functioning via the modulation of T cell activity.

The present invention also encompasses therapeutic methods for the treatment of a variety of immune disorders with the use of the altered DC. In embodiments of the invention, the DC may be transfected in vitro to produce a desired DC phenotype and then either used ex vivo or alternatively used in vivo as administered to a mammalian subject.

According to an aspect of the present invention there is provided a mammalian immune cell that exhibits a targeted gene-specific knockout phenotype, said immune cell being capable of altering an immune response in a-mammal via the modulation of T cell activity. In embodiments, the immune cell may be selected from an endothelial cell or an antigen presenting cell (APC). In more preferred embodiments, the immune cells is an APC selected from the group consisting of DC, macrophages, myeloid cells, B lymphocytes and mixtures thereof.

According to an aspect of the invention is a mammalian immune cell exhibiting a targeted endogenous gene-specific knockout phenotype, said immune cell altering an immune response in a mammal via the modulation of T cell activity

According to another aspect of the present invention is a mammalian immune cell that exhibits a targeted gene-specific knockout phenotype, wherein said gene is selected from one or more of a surface marker, a chemokine, a cytokine, an enzyme and a transcriptional factor.

According to another aspect of the present invention is an APC which does not express one or more of a surface marker, a chemokine, a cytokine, an enzyme and a transcriptional factor. In an embodiment of the invention, the APC is a DC.

According to another aspect of the present invention is a dendritic cell (DC) which contains at least one double-stranded RNA molecule capable of inhibiting the expression of an endogenous target gene encoding a molecule selected from the group consisting of a surface marker, a chemokine, a cytokine, an enzyme, a transcriptional factor and combinations thereof.

According to another aspect of the present invention is a tolerogenic dendritic cell (DC) which contains at least one double-stranded RNA molecule capable of inhibiting the expression of IL-12.

According to a further aspect of the invention is the use of a mammalian immune cell that exhibits a targeted gene-specific knockout phenotype, wherein said gene is selected from one or more of a surface marker, a chemokine, a cytokine, an enzyme and a transcriptional factor, in a medicament for the treatment of an immune disorder characterized by inappropriate T cell activity.

According to another aspect of the invention is the use of a siRNA possessing specific homology to part or the entire exon region of a gene encoding a surface marker, a chemokine, a cytokine, an enzyme or a transcriptional factor of an antigen presenting cell (APC), in a medicament for the treatment of an immune disorder characterized by inappropriate T cell activity.

According to yet another aspect of the invention is a composition for the treatment of an immune disorder, said composition comprising at least one of:
(a) a construct that inhibits the expression of an endogenous target gene encoding a surface marker, a chemokine, a cytokine, an enzyme or a transcriptional factor in an immune cell such that said immune cell alters T cell activity; and
(b) an immune cell wherein said immune cell comprises at least one construct that inhibits the expression of an endogenous target gene encoding a surface marker, a chemokine, a cytokine, an enzyme or a transcriptional factor, and
(c) a pharmaceutically acceptable carrier,
wherein said composition alters T cell activity leading to an altered immune response.

According to another aspect of the invention is a method for inhibiting the T cell activating ability of a DC, the method comprising transforming said DC with a constructcapable of inhibiting-the expression of an endogenous target gene encoding a surface marker, a chemokine, a cytokine, an enzyme or a transcriptional factor.

According to still a further aspect of the invention is a method for decreasing the immunogenicity and rejection potential of an organ for transplantation, said method comprising perfusing said organ with a composition that suppresses T cell activity, said composition comprising at least one construct that inhibits the expression of an endogenous target gene encoding a surface marker, a chemokine, a cytokine, an enzyme or a transcriptional factor and a pharmaceutically acceptable carrier.

According to another aspect of the invention is a method for making an immune cell that alters the activity of T cells in vivo, said method comprising;

transforming immune cells in vitro with at least one construct that inhibits the expression of an endogenous target gene encoding a surface marker, a chemokine, a cytokine, an enzyme or a transcriptional factor.

According to yet another aspect of the invention is method for the treatment of autoimmune disorders and transplantation rejection in a mammalian subject, said method comprising administering a therapeutically effective amount of a composition to said subject, said composition comprising DC that contain at least one construct that inhibits the expression of an endogenous target gene encoding a surface marker, a chemokine, a cytokine, an enzyme or a transcriptional factor, wherein said DC suppresses T cell activity.

According to another aspect of the invention is a method for the treatment of autoimmune disorders and transplantation rejection in a mammalian subject, said method comprising administering a therapeutically effective amount of a composition to said subject, said composition comprising an siRNA targeted to inhibit expression of an endogenous target gene in an antigen presenting cell, said gene encoding a surface marker, a chemokine, a cytokine, an enzyme or a transcriptional factor, wherein said siRNA suppresses T cell activity.

In aspects of the invention the construct may be any suitable construct that can be used to target and silence a particular gene of interest. In embodiments, the construct is siRNA or hybrid DNA/RNA provided alone or within a suitable vector or plasmid.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

Preferred embodiments of the present invention will now be described more fully with reference to the accompanying drawings:

FIG. 1 shows the efficacy of DC siRNA transfection. Day 7 bone marrow derived DC (1×10⁶) were transfected with unlabeled control siRNA (Ctrl-siRNA, left), or fluorescein labelled siRNA specific for luciferase GL2 duplex (Fl-siRNA, middle) at 60 pM concentration. Fl-siRNA was also added to day 4-cultured DC without transfection reagents (Phagocytosis, right). DC were activated with LPS/TNFα on day 8 and the transfection efficacy was assessed by flow cytometry on day 9. Data are representative of three independent experiments.

FIG. 2 shows that DC viability is not affected by siRNA transfection. DC cultured from bone marrow progenitors and 1×10⁶ day-7 immature DC were left untreated or were transfected with GenePorter alone, siRNA-IL12p35 alone, or the combination of both for 48 hrs. Percentage apoptosis and necrosis was assessed using annexin-V and propidium iodine (PI), respectively, by flow cytometry. Data are representative of three independent experiments.

FIG. 3 shows that siRNA transfection of DC does not alter nor induce DC maturation. In panel 3A immature DC (1×10⁶) were cultured alone (untransfected), pre-treated for 24 hrs with GenePorter (mock transfected), or transfected with 60 pM siRNA-IL12p35. The transfected DC were subsequently activated for 24 hrs with 10 ng/ml LPS and 10 ng/ml TNF-α. Maturation was assessed by expression of CD11c, MHC II, CD40, and CD86 by flow cytometry using FlTC-conjugated antibodies (solid line), and isotype controls (broken line). In panel 3B immature DC (1×10⁶) were untreated (untransfected), treated with GenePorter alone (mock transfected) or transfected with 60 pM siRNA-IL12p35 for 24 hrs at which time maturation was assessed by expression of CD11c, MHC II, CD40, and CD86 by flow cytometry using FITC-conjugated antibodies (solid line), and isotype controls (broken line). Data are representative of three independently performed experiments.

FIG. 4 shows the specificity of gene inhibition by siRNA. DC (1×10⁶) were transfected with 60 pM siRNA-IL12p35, siRNA-IL12p40 or Geneporter alone (mock transfected). The transfected DC were activated with 10 ng/ml LPS and 10 ng/ml TNF-α for 24 hrs. RNA from the treated DC was extracted by the Trizol method. RT-PCR was performed to assess expression of IL-12p35, IL-12p40 and GAPDH using primers described in the examples section. Data are representative of three independent experiments.

FIG. 5 shows that siRNA-IL12p35 transfection of DC specifically blocks IL-12 and upregulates IL-10. DC (1×10⁶) were unmanipulated (control), transfected with Geneporter alone (mock transfected), transfected with 60 pM siRNA-IL12p35, or 60 pM siRNA-IFNγ (siRNA control). The transfected DC were activated with 10 ng/ml LPS and 10 ng/ml TNFα for 24 hrs. In panel 5A the supernatants were harvested from cultures and analyzed for IL12 p70 production using ELISA. In panel 5B the supernatants were harvested from cultures and analyzed for IL-10 production using ELISA. Data represent mean+SEM and are representative of three experiments (*, p<0.01; by one-way ANOVA and Newman-Keuls test).

FIG. 6 shows that siRNA-IL12p35 transfection inhibits DC allostimulatory ability. C57BL/6 derived DC (1×10⁶) were untreated (untransfected, 0), transfected with GenePorter alone (mock transfected, 0), transfected with 60 pM siRNA-IFNγ (control siRNA, Δ) or transfected with 60 pM siRNA-IL12p35 (•) for 24 hrs. Allogeneic (BALB/c) T cells (2×10⁵/well) were incubated with siRNA-treated DC at the indicated numbers for 72 hrs. Proliferation was determined using [³H]-thymidine incorporation. Data are representative of three independent experiments. (*p<0.01; by one-way ANOVA and Newman-Keuls test).

FIG. 7 shows that siRNA-IL12p35-transfected DC promote Th2 polarization. In panel 7A C57/BL6 bone marrow derived DC were pretreated with GenePorter alone (mock transfected), transfected with 60 pM siRNA-lL12p35 for 24 hrs. Subsequently siRNA-treated DC (10⁶) were cultured with allogeneic (BALB/c) T cells (10×10⁶) for 48 hrs. T cells were purified from co-culture using a T cell column and RT-PCR was performed for IL-4, IFN-γ, and GAPDH. In panel 7B C57/BL6 bone marrow derived DC were unmanipulated (control), pretreated with GenePorter alone (mock transfected), transfected with 60 pM siRNA-IL12p35, or 60 pM siRNA-IFN-γ (siRNA control) for 24 hrs. siRNA-treated DC (10⁶) were subsequently cultured with allogeneic (BALB/c) T cells 10×10⁶) for 48 hrs. Supematants were collected from the cultures and IFN-γ (Th1 cytokine) and IL-4 (Th2 cytokine) production was assessed by ELISA. (*p<0.01; by one-way ANOVA and Newman-Keuls test).

FIG. 8 shows that siRNA-IL12p35-treated DC stimulate antigen-specific Th2and inhibit Th1 responses in vivo. Day 7 bone marrow derived DC cultured in GM-CSF and IL-4 were transfected with IL12p35-siRNA, or mock transfected. Subsequently cells were pulsed with 10 μg/ml of KLH for 24 hrs and injected subcutaneously (5×10⁵ cells/mouse) into syngeneic C57BL/6 mice. After 10 days, T cells from lymph nodes were isolated from recipient mice. A KLH-specific recall response was performed as described in the example section. IFN-γ and IL-4 response to KLH was assessed by ELISA. Data shown are pooled from 3 independent experiments.

### DETAILED DESCRIPTION OF THE PREFERRED EMBODIMENTS

The present invention provides transformed immune cells that exhibit a gene specific targeted knock-out phenotype. Such transformed immune cells can be used in a variety of therapeutic in vitro, ex vivo and in vivo methods to modulate T cell activity and thus have use in therapeutic approaches for the treatment of immune disorders in mammalian subjects.

The immune cells of the invention exhibit a targeted gene-specific knockout phenotype which imay be accomplished using any technique that provides for the targeted silencing of an endogenous gene. In one aspect of the invention the technique of RNAi (RNA interference) was used to create transformed immune cells suitable for use for the modulation of T cell activity in vitro, ex vivo or in vivo. In this aspect, the immune cells are transfected with a siRNA (small interfering RNA) designed to target and thus to degrade a desired mRNA in order not to express the encoded protein that is involved in T cell activity. Thus such transfected immune cells may be used to suppress or stimulate immune system functioning via the modulation of T cell activity. It is understood by those of skill in the art that any method for silencing a specific gene may be used in the present invention. Representative examples of suitable techniques include but are not limited to RNAi and hybrid DNA/RNA constructs. The hybrid DNA/RNA constructs are essentially siRNA constructs in which the nucleic acid composition used for silencing is altered to include DNA (Lamberton J. and Christian A. 2003. Mol. Biotechnol. June;24(2):111-20, the entirety of the disclosure is incorporated herein by reference).

It is desirable to modulate T cell activity, ie. suppress T cell activity in a variety of immune disorders selected but not limited to the group consisting of septic shock, rheumatoid arthritis, transplant rejection, scleroderma, immune mediated diabetes, chronic inflammatory bowel syndrome, HIV, cancer, colitis, Crohn's disease, Goodpasture's syndrome, Multiple Sclerosis, Grave's disease, Hashimoto's thyroditis, Autoimmune pernicious anemia, Autoimmune Addison's disease, Vitiligo, Myasthenia gravis, Scleroderma, Systemic lupus erythematosus, Primary Sjogren's syndrome, Polymyositis, Pemphigus vulgaris, Ankylosing spondylitis, Acute anterior uveitis, Hypoglycemia and inflammation associated with chronic illness. Thus the siRNA, transfected immune cells and compositions containing such can be used in methods to treat the aforementioned immune disorders by the down regulation of T cell activity leading to a prevention or decrease in an autoimmune response and prevention of tissue/organ rejection.

Immune cells for use in the present invention may be selected from antigen presenting cells (APC) and endothelial cells. Both APC and endothelial cells (Limmer A., et al., 2001. Arch Immunol Ther Exp (Warsz). Suppl 1:S7-11; Perez V/L., et al., 1998. Cell Immunol. October 10;189(1):31-40) are known to be able to activate T cells. In preferred embodiments of the invention, the immune cells are APC that may be selected from the group consisting of macrophages, myeloid cells, B lymphocytes, DC and mixtures thereof. It is also within the scope of the present invention to use other APC capable of activating T cells through the T cell receptor as is understood by one of skill in the art. In particularly preferred embodiments of the invention, the immune cell is a DC. APC such as DC are known to be phagocytic in nature and thus tend to take up molecules within their environment. In the present invention DC is specifically demonstrated to be successfully altered with siRNA to exhibit a stable phenotype. Therefore one of skill in the art would readily understand that any APC may be altered in accordance with the present invention and used in the methods of the invention. It is also understood that a combination of different types of immune cells may be used in the methods of the present invention.

According to an embodiment of the invention, DC are transformed with a designed siRNA. In this embodiment DC must be isolated from a subject and expanded in vitro. DC are typically derived from a source such as bone marrow, peripheral blood, spleen and lymph. Blood is the preferred source of DC because it is readily accessible and may be obtained in large quantities. Substances which stimulate hematopoiesis (i.e. G-CSF and GM-CSF) may be first administered to the subject in order to increase the number of DC. Blood is treated to isolate the DC from other cell types by standard methods known in the art. Isolated DC cultured in vitro may be treated with cytokines to increase their number. Methods for isolating and ex vivo culture of DC are known in the art and described for example in U.S. Pat. Nos. 5,199,942, 5,851,756, 6,017,527, 6,251,665, 6,458,585 and 6,475,483 (the disclosures of which are incorporated herein by reference in their entirety).

The present invention also encompasses therapeutic methods for the treatment of a variety of immune disorders in a mammalian subject. The methods may involve the use of a siRNA designed for use directly in vivo to block the expression of a gene by an immune cell, the gene expressing a protein involved in the activity of T cells which elicits an immune disorder. Alternatively, the methods may involve the use of an immune cell which contains at least one double-stranded RNA molecule (siRNA) that inhibits the expression of an endogenous target gene encoding a surface marker, a chemokine, a cytokine, an enzyme or a transcriptional factor. In preferred embodiments of the invention, the methods of the invention comprise the use of an altered (i.e. transformed) DC that contains a double-stranded RNA molecule that inhibits the expression of an endogenous target gene encoding a surface marker, a chemokine, a cytokine, an enzyme or a transcriptional factor. Still in other embodiments, the therapeutic method may involve ex vivo treatment of tissues and/or organs intended for transplantation. In aspects of the invention, the siRNA possesses specific homology to part or to the entire exon region of a surface marker, a chemokine, a cytokine, an enzyme or a transcriptional factor normally expressed by the immune cell such that the gene is silenced

It is understood by one of skill in the art that the siRNA as herein described may also include altered siRNA that is a hybrid DNA/RNA construct or any equivalent thereof.

In preferred embodiments of the invention the transfected DC cells are prepared by the method of RNAi. RNA interference is a mechanism of post-transcriptional gene silencing. Specific gene silencing is mediated by short strands of duplex RNA of approximately 21 nucleotides in length (termed small interfering RNA or siRNA) that target the cognate mRNA sequence for degradation. While many techniques have been used to block specific molecules in vitro and in vivo, such as anti-sense oligonucleotides (Gerwitz, A. M. 1999. Curr Opin Mol Ther 1:297) and monoclonal antibodies (Drewe, E., et al., 2002. J Clin Pathol 55:81), RNAi was used in the present invention because it provides several distinct advantages. First, mRNA degradation by siRNA is extremely efficient as only a few copies of dsRNA are necessary to activate the RNA Induced silencing complex (RISC) (Martinez, J. A. et al., 2002. Cell 10:563). Once RISC is activated it can conduct multiple rounds of gene-specific mRNA cleavage. Second, RNAi is specific, in that only sequences with identity to one of the strands of dsRNA will be cleaved (Hannon, G. J. 2002. Nature 418:244). Third, the RNAi effect is long lasting and can be spread to progeny cells after replication, although a dilution effect is evident in mammalian cells (Fire, A., et al., 1998. Nature 391:806). This technique is relatively simple, giving rise to an in vitro knock down phenotype within days that can be confirmed with many antibody based detection systems (such as ELISA or Western Blotting), or if an antibody is not available, by RT-PCR or functional assays.

DC may be transformed with siRNA alone, siRNA contained within a plasmid or vector that results in the production of the siRNA, siRNA contained within a plasmid or vector that further expresses a selected antigen and siRNA together with a mRNA from a tumor cell. In the case of the plasmid or is vector further expressing a selected antigen, the DC will process or modify the antigen in a manner-to promote the stimulation of T cell activity by the processed or modified antigens. Methods for making siRNA and cell transformation are described for example in U.S. Patent Application 2002/0173478, U.S. Patent Application 2002/0162126, PCT/US01/10188, PCT/EP01/13968 and in Simeoni F., et al., 2003 Nucleic Acids Res June 1;31(11):2717-24 (the disclosures of which are incorporated herein in their entirety). Methods for producing antigen pulsed DC are known and exemplified for example in U.S. Pat. No. 6,497,876 and U.S. Pat. No. 6,479,286 (the disclosures of which are incorporated herein by reference in their entirety). Methods for making siRNA plasmids or vectors are also known and described for example in U.S. Patent Application 2003/0104401, in Morris M. C., et al., 1997. Nucleic Acid Res. July 15:25(14):2730-6 and in Van De Wetering M., et al., 2003, EMBO June;4(6):609-15 (the disclosures of which are incorporated herein in their entirety). Suitable lipid-based vectors may include but are not limited to lipofectamine, lipofectin, oligofectamine and GenePorter™. Methods for producing tumor derived RNA for pulsing DC are also known to those of skill in the art and are described for example in U.S. Patent Application 2002/0018769 (the disclosure of which is incorporated herein in its entirety).

In embodiments of the invention, DC are transformed to contain a double-stranded RNA molecule that inhibits the expression of an endogenous target gene encoding a protein that either suppresses T cell activation or alternatively stimulates T cell activation. For the suppression of T cell activation, the immune cells of the invention are transformed with a double-stranded RNA molecule that inhibits the expression of a gene that encodes a co-stimulatory molecule, cytokine, adhesion molecule, enzyme or transcription factor. Representative examples of such co-stimulatory molecules, cytokines, adhesion molecules, enzymes and transcription factors may be selected from the group consisting of TNFα, IL-1, IL-1b, IL-2, TNFβ, IL-6, IL-7, IL-8, IL-23, IL-15, lL18, IL-12, IFNγ, IFNα, lymphotoxin, DEC-25, CD11c, CD40, CD80, CD86, MHCI, MHCII, ICAM-1, TRANCE, CD200, CD200 receptor, CD83, CD2, CD44, CD91, TLR-4, TLR-9, 4-1 BBL, nicotinic receptor, GITR-L, OX-40L, CD-CK1, TARC/CCL17, CCL3, CCL4, CXCL9, CXCL10, IKK-β, NF-κB, STAT4, ICSBP/IFN, regulatory factor 8, TRAIL, Inos, arginase, FcgammaRI and II, thrombin, MIP-1α and MIP-1B.

For the activation of T cells where such activation is desired, the immune cells of the invention are transformed with a double-stranded RNA molecule that inhibits the expression of a gene encoding a surface marker or enzyme that suppresses T cell activation. Representative examples of such surface markers and enzymes may be selected from the group consisting of B7-H1, EP2, IL-10 receptor, VEGF-receptor, CD101, PD-L1, PD-L2, HLA-11, DEC-205, CD36 and indoleamine 2,3-dioxygenase. It may be desirable to activate T cells in a variety of conditions associated with immune suppression such as but not limited to cancer, HIV and parasitic infections. Where immune suppression is present, it is desirable to use the cells and methods of the invention to increase T cell activity leading to an enhanced immune response (Curiel T. J., et. Al., 2003. Nat Med May;9(5):562-7).

It is within the scope of the invention to transform a selected immune cell with more than one double-stranded RNA molecule (an siRNA) or hybrid DNA/RNA in order to simultaneously inhibit the expression of more than one endogenous gene normally expressed by the immune cell. The number of double-stranded RNA molecules transformed into any given immune cell being dependent on the resultant extent of inhibition of the expression of the target gene which is readily determined as is understood by one of skill in the art.

In the present invention in one embodiment, the induction of RNAi in DC was conducted using siRNA specific for IL-12 p35 (siRNA-IL12p35). It was demonstrated that bioactive IL-12 p70 production in bone marrow-derived DC was inhibited after stimulation with LPS and TNF-α, and was accompanied by an increase in IL-10 production. Moreover, when siRNA-IL12p35-treated DC were cultured with allogeneic T cells, a Th2 polarization was observed since T cell expression of IFN-γ was reduced while IL-4 was increased. Inhibiting IL-12 production using siRNA-IL12p35 was associated with suppressed DC allostimulatory function. In vivo, initiation of antigen-specific Th2 responses was observed when DC treated with siRNA-IL12p35 were pulsed with KLH and used for immunization experiments. Overall these results demonstrate for the first time that RNAi can be induced in DC and that siRNA is a potent tool for modulating DC function and subsequently T cell polarization.

DC are Efficiently Transfected with siRNA

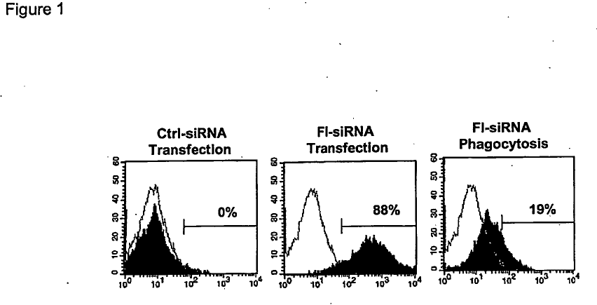

To establish a protocol for RNAi in DC, the siRNA-transfection efficacy was first assessed. Many studies have shown a limited ability of DC to be transfected with DNA. To determine the transfection efficacy, fluorescein labelled siRNA was synthesized that is specific for luciferase (FL-siRNA-Luc), a gene that does not exist in mammalian cells and thus does not affect cellular function. siRNA lacking fluorescein (siRNA-Luc) was used as a non-labelled control. FL-siRNA-Luc and siRNA-Luc were transfected by GenePorter into bone marrow-derived and cultured DC. After 24 hrs siRNA transfection, the percentages of DC that had incorporated FL-siRNA-Luc were quantified by flow cytometry. As seen in FIG. 1, FL-siRNA-Luc had been successfully incorporated into 88% of the cells, as analyzed by flow cytometry.

It was then assessed whether immature DC are able to internalize naked siRNA. Immature DC on day 4 were cultured with FL-siRNA-Luc in the absence of transfection reagent, and assessed for siRNA internalization by flow cytometry on day 9 of culture. Despite the long incubation period, 19% of DC still contained incorporated siRNA (FIG. 1), suggesting that naked siRNA may be used for transfection of DC.

siRNA Transfection does not Alter DC Viability, Maturation or Phenotvpe

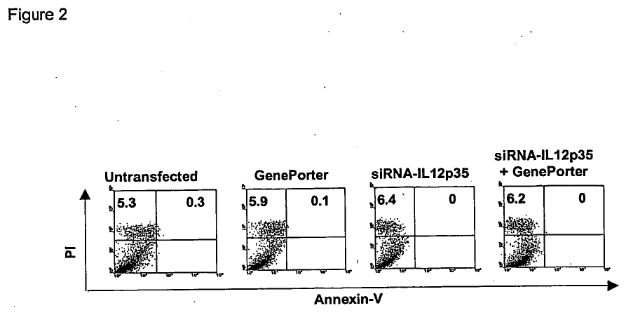

One of the major concerns for gene transfection is that transfection reagents may affect cellular function or viability. Although a high level of transfection efficiency was already demonstrated using the GenePorter method, it was further needed to establish whether siRNA or the transfection procedure itself altered the viability of the DC. Thus, day-7 bone marrow-derived DC were treated with transfection reagent (GenePorter) alone, siRNA-lL12p35 alone, or the combination of transfection reagent and siRNA-IL12p35. After 24 hrs of transfection, apoptosis and necrosis was assessed using annexin-V and propidium iodine (PI) staining respectively. Compared to untreated DC, neither the transfection protocol alone, nor the siRNA affected cell viability (FIG. 2).

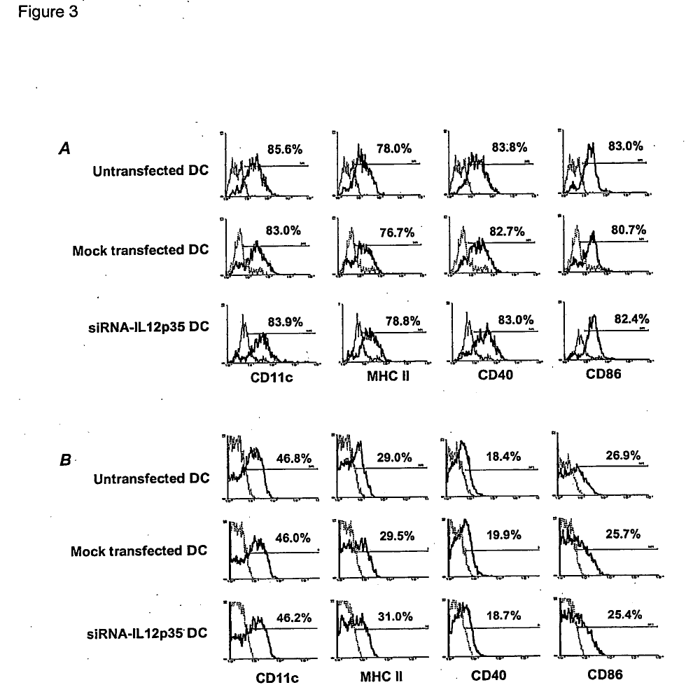

Next it was addressed whether the siRNA or the transfection procedure affected DC maturation. DC were transfected with siRNA following activation with LPS and TNF-α. DC maturation was assessed by flow cytometry to analyze expression of MHC II, CD40, and CD86 or the DC-specific marker CD11c. It can be seen that neither treatment with siRNA nor mock transfection altered DC maturation in response to LPS and TNF-α (FIG. 3A).

An additional concern associated with transfecting DC with nucleic acids is induction of maturation. Since long double stranded RNA (poly(I):poly(C)) has previously been shown to induce maturation and activation of immature DC (25), it was determined whether or not siRNA had the same effect. Thus, immature DC were treated with siRNA-IL12p35 for 24 hrs and cell surface maturation markers were assessed by FACS. FIG. 3B illustrates that siRNA treatment alone failed to upregulate MHC II, CD40, or CD86 on immature DC. Although these experiments used a concentration of 60 pM of siRNA-IL12p35, higher concentrations of siRNA-IL12p35 (up to 10 fold) were also assessed, with no alteration in viability or differentiation (data not shown). These data indicate that transfection of DC with siRNA-IL12p35 affects neither the viability nor phenotype.

siRNA induces Specific Gene Silencing in DC

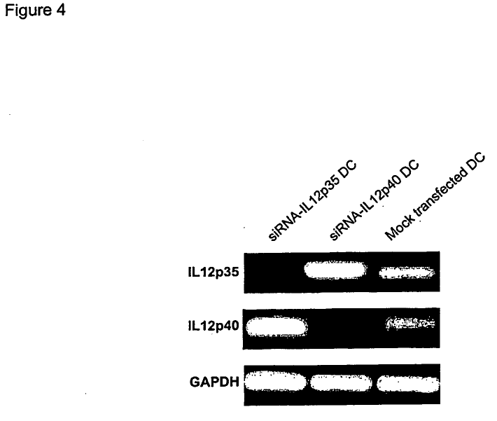

The specificity of siRNA induced gene silencing in DC was examined by transfecting DC with siRNA-IL12p35 and siRNA targeted to the p40 component of IL-12 (siRNA-IL12p40). Transcripts of IL-12 p35 and IL-12 p40 were detected by RT-PCR using primers flanking the siRNA targeted sequence. Specific inhibition was demonstrated at the transcript level: siRNA-IL12p35 exclusively suppressed p35 transcripts while siRNA-IL12p40 suppressed only p40 transcripts (FIG. 4). In addition, both siRNA-IL12p35 and siRNA-IL12p40 failed to affect transcripts of the house-keeping gene GAPDH. These data suggested that siRNA-mediated gene silencing is specific in DC.

siRNA-IL12p35 Inhibits IL-12 Expression in DC

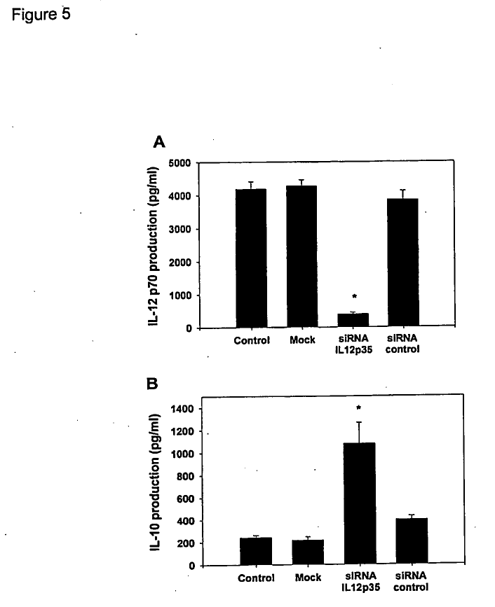

It was verified whether siRNA-IL12p35 can block production of IL-12 protein. Since IL-12p35 is critical for the formation of the IL-12 p70 heterodimer, the production of this cytokine was assessed in the supernatant of LPS/TNF-α-activated DC using ELISA. DC transfected with siRNA-IL12p35 were stimulated with LPS and TNF-α for 48 hrs to induce maturation and cytokine expression. To confirm specificity of gene silencing, siRNA specific for IFN-γ (siRNA-control) was used since this cytokine is not expressed in bone marrow derived DC. Additionally, negative controls included DC transfected with GenePorter alone (mock transfected DC) and unmanipulated DC (untreated control). As shown in FIG. 5A, siRNA-IL12p35 reduced IL-12p70 heterodimer production (as determined by ELISA) by 85-90% compared to untreated or mock transfected DC. More importantly this effect was specific since no significant difference in IL-12p70 production was seen in DC treated with the IFN-γ siRNA-control. In addition, levels of IL-10 production were tested since a reciprocal relationship with IL-12 production has been previously reported (27). IL-10 production in DC treated with siRNA-IL12p35 was significantly and specifically upregulated compared to controls (FIG. 5B).

siRNA-IL12D35 Suppresses DC Allostimulatory Activity

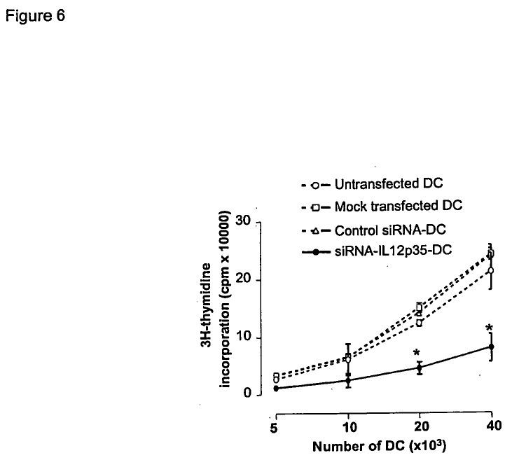

DC function can be characterized in part by their ability to stimulate alloreactive T cells in the mixed lymphocyte reaction (MLR) (8). To determine whether siRNA-IL12p35 affected DC allostimulatory activity, MLR was performed using DC transfected with siRNA-IL12p35, siRNA-control, mock transfected, or untreated controls. Allogeneic T cells were cultured with siRNA-transfected DC for 48 hrs at which point allostimulation was determined by proliferation. While the control DC groups all showed similar allostimulatory activity, DC transfected with siRNA-IL12p35 significantly suppressed this response (FIG. 6).

siRNA-lL12p35 Treated DC Promote Th2 Differentiation

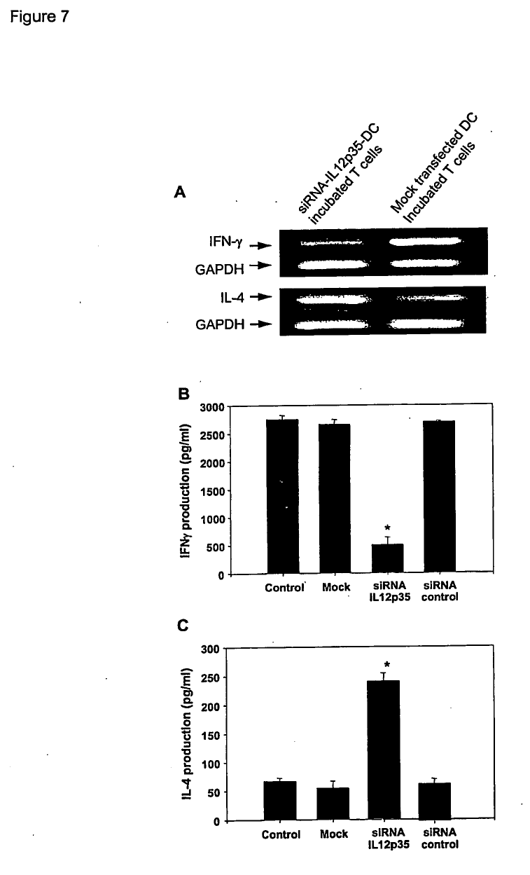

Since IL-12p70 is a key cytokine responsible for polarizing T cells towards an IFN-γ-producing or Th1 phenotype (Trinchieri, G. 1998. Adv Immunol 70:83), it was assessed whether allostimulation with DC that were transfected with siRNA-IL12p35 could alter cytokine production from responding T cells. Mock transfected DC stimulated high IFN-γ and low IL4 mRNA transcripts from responding T cells, however, stimulation with siRNA-IL12p35 treated DC resulted in low IFN-γ and high IL-4 transcripts (FIG. 7A). To confirm these results at the protein level IFN-γ and IL-4 were assayed from MLR culture supernatants using ELISA. The T cells incubated with siRNA-IL12p35-treated DC produced low levels of IFN-γ (FIG. 7B) and high levels of IL-4 (FIG. 7C). In contrast, T cells incubated with untransfected DC, GenePorter transfected DC or DC transfected with control siRNA showed a cytokine profile of high IFN-α and low IL-4. These data suggest that siRNA-IL12p35-treated DC have the ability to polarize naïve T cells along the Th2 pathway.

Modulation of Antigen-Specific Response In Vivo using siRNA-IL12p35 Treated DC

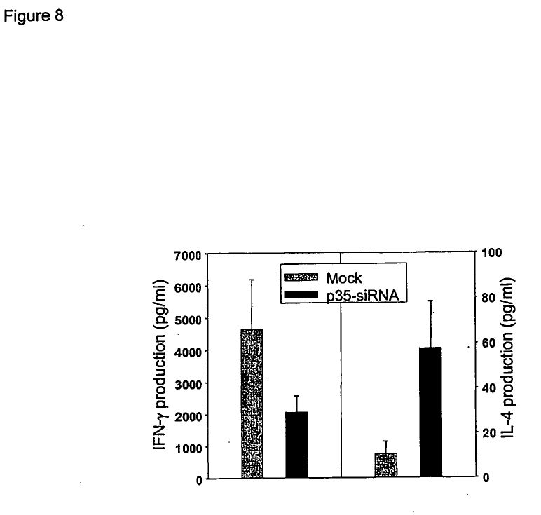

Although a shift from Th1 cytokine production to Th2 is seen when naïve T cells are incubated with siRNA-IL12p35-treated DC, it was investigated whether this effect could also be obtained in vivo. To accomplish this, siRNA-IL12p35-treated or mock transfected DC with KLH were transfected and used as immunogens in vivo by injecting into syngeneic hosts. Ten-days after immunization with KLH-pulsed control DC, a Th1 recall response was evident when draining lymph node cells from recipient mice were challenged with KLH in vitro, as determined by upregulated IFN-γ and downregulated IL-4 production (FIG. 8). Under the same conditions the siRNA-IL12p35-treated DC promoted a Th2 shift in the recall cytokine response, showing increased IL-4 production and suppressed IFN-γ. These results suggest that antigen-pulsed and siRNA-modified DC can be used to modulate the Th1 vs Th2 balance in vivo during a primary immune response.

Interestingly, DC silenced by siRNA-IL12p35 showed decreased allostimulatory capacity which is in contrast to results reported using DC generated from IL-12 knockout mice that possess normal allostimulatory activity (Piccotti, J. R., et al., 1998. J Immunol 160:1132; Tourkova I. L., et al., 2001. Immunol Lett 78:75). We attribute this discrepancy to compensatory immunological mechanisms that may have arisen in the lifetime of the IL-12 knockout mice. This is suggested by studies that have demonstrated the importance of IL-12 in MLR. First, IL-12 production by antigen presenting cells was demonstrated to be critical for MLR proliferative response since addition of anti-IL-12 antibodies resulted in suppression of proliferation (Kohka, H., et al., 1999. J Interferon Cytokine Res 19:1053). Second, overexpression of IL-12 in DC results in increased allostimulatory function (Kelleher, P., et al., 1998. Int Immunol 10:749). Another possible explanation for suppressed MLR in siRNA-IL12p35-transfected DC is that the increased IL-10 production may act as an inhibitor of T cell proliferation (Wang X. N., et al., 2002. Transplantation 74:772; Tadmori W., et al., 1994. Cytokine 6:462). Other studies examining naturally occurring Th2-promoting DC have shown that these cells have a reduced allostimulatory function and reduced IL-12 production (Gao J. X., et al., 1999. Immunology 98:159; Khanna A., et al., 2000. J Immunol 164:1346). The combination of Th2 promoting properties, as well as poor allostimulation suggests that siRNA-IL12p35 transfected DC may possess the phenotype of a “tolerogenic” DC and thus may be useful for treatment of Th1 mediated autoimmune diseases and transplant rejection.

The present invention provides methods of using therapeutic compositions comprising siRNA designed to target a specific mRNA as well as activated and nonactivated altered (i.e.transformed) immune cells that contain the siRNA in embodiments as described supra. A feature of DC is their capacity to migrate or home to T-dependent regions of lymphoid tissues where DC may affect T cell activity and elicit a modulated immune response. Therefore, in vivo administration of a siRNA composition would be effective in targeting and having a modulatingeffect on T cell activity.

In one embodiment, the compositions comprise DC containing siRNA specifically designed to degrade mRNA encoding a surface marker, a chemokine, a cytokine, an enzyme or a transcriptional factor such that the transformed DC leads to a lack of expression of the surface marker, chemokine, cytokine, enzyme or transcriptional factor and as a result affect the activity of T cells to modulate an immune response. Such DC may be provided as compositions for administration to a mammalian subject or as compositions for ex vivo approaches for the treatment of cells, tissues and/or organs for transplantation. Such compositions may contain pharmaceutically acceptable carriers or excipients suitable for rendering the mixture administrable orally or parenteraly, intravenously, intradermally, intramuscularly or subcutaneously or transdermally. The transformed immune cells or siRNA may be admixed or compounded with any conventional, pharmaceutically acceptable carrier or excipient as is known to those of skill in the art.

As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations.

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

It is also understood by one of skill in the art that the compositions of the invention may be provided on a device for in vitro, ex vivo or in vivo use. Suitable structures may include but are not limited to stents, heart valves, implants and catheters.

The therapeutically effective amount of active agent to be included in the pharmaceutical composition of the invention depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg.

While it is possible for the agents to be administered as the raw substances, it is preferable; in view of their potency, to present them as a pharmaceutical formulation. The formulations of the present invention for mammalian subject use comprise the agent, together with one or more acceptable carriers therefor and optionally other therapeutic ingredients. The carrier(s) must be “acceptable” in the sense of being compatible with the other ingredients of the formulation and not deleterious to the recipient thereof. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier, which constitutes one or more accessory ingredients. In general, the formulations are prepared by uniformly and intimately bringing into association the agent with the carrier(s) and then, if necessary, dividing the product into unit dosages thereof.

Formulations suitable for parenteral administration conveniently comprise sterile aqueous preparations of the agents, which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization.

Such formulations may optionally contain one or more additional ingredients among which may be mentioned preservatives, such as methyl hydroxybenzoate, chlorocresol, metacresol, phenol and benzalkonium chloride. Such materials are of special value when the formulations are presented in multidose containers.

Compositions of the invention comprising a selected targeting siRNA can also comprise one or more suitable adjuvants. In this embodiment siRNA can be used as a vaccine in order to stimulate or inhibit T cell activity and polarize cytokine production by these T cells. As is well known to those of ordinary skill in the art, the ability of an immunogen to induce/elicit an immune response can be improved if, regardless of administration formulation (i.e. recombinant virus, nucleic acid, peptide), the immunogen is coadministered with an adjuvant. Adjuvants are described and discussed in “Vaccine Design—the Subunit and Adjuvant Approach” (edited by Powell and Newman, Plenum Press, New York, U.S.A., pp. 61-79 and 141-228 (1995). Adjuvants typically enhance the immunogenicity of an immunogen but are not necessarily immunogenic in and of themselves. Adjuvants may act by retaining the immunogen locally near the site of administration to produce a depot effect facilitating a slow, sustained release of immunizing agent to cells of the immune system. Adjuvants can also attract cells of the, immune system to an immunogen depot and stimulate such cells to elicit immune responses. As such, embodiments of this invention encompass compositions further comprising adjuvants.

Desirable characteristics of ideal adjuvants include:
1) lack of toxicity:
2) ability to stimulate a long-lasting immune response;
3) simplicity of manufacture and stability in long-term storage;
4) ability to elicit both cellular and humoral responses to antigens administered by various routes, if required:
5) synergy with other adjuvants;
6) capability of selectively interacting with populations of antigen presenting cells (APC);
7) ability to specifically elicit appropriate Tr, TR1 or TH2 cell-specific immune responses; and
8) ability to selectively increase appropriate antibody isotype levels (for example, IgA) against antigens/immunogens.

Suitable adjuvants include, amongst others, aluminium hydroxide, aluminium phosphate, amphigen, tocophenols, monophosphenyl lipid A, muramyl dlpeptide and saponins such as Quill A. Preferably, the adjuvants to be used in the tolerance therapy according to the invention are mucosal adjuvants such as the cholera toxine B-subunit or carbomers, which bind to the mucosal epithelium. The amount of adjuvant depending on the nature of the adjuvant itself as is understood by one of skill in the art.

Compositions of siRNA of the present invention may also be provided within antibody labelled liposomes (immunoliposomes) or antibody-double stranded RNA complexes. In this aspect, the siRNA is specifically targeted to a particular cell or tissue type to elicit a localized effect on T cell activity. Specifically, the liposomes are modified to have antibodies on their surface that target a specific cell or tissue type. Methods for making of such immunoliposomal compositions are known in the art and are described for example in Selvam M. P., et.al., 1996. Antiviral Res. December;33(1):11-20 (the disclosure of which is incorporated herein in its entirety).

In one representative embodiment of the invention, siRNA to TNFα is made according to the methods of Tuschl T., et al., 1999. Genes Dev. 13:3191-97 and Tuschl T., et.al., 1998. EMBO J. 17:2637-2650. In these methods, 21 nucleotide base-pair sequences are chemically synthesized using a new 5′-silyl protecting group in conjunction with a unique acid-labile 2′-orthoester protecting group, 2′-bis(acetoxyethoxy)-methyl ether (2′-ACE). The 2′-protecting groups are rapidly and completely removed under mild conditions in aqueous buffers. This “2′-ACE™ technology (Dharmacon Inc. CO, USA) enables the synthesis of RNA oligonucleotides in high yield. To the siRNA specific to TNFα is admixed an agent that crosses the cell membrane and enters the nucleus in order to achieve maximal inhibition of TNFα. Such agents are known to those of skill in the art and may be selected from cationic and anionic liposomes as well as compositions of chemicals which permit transmembrane entrance of the siRNA without affecting the function of the nucleotides. In addition to compounds which allow entry of siRNA into the cell, the siRNA may be mixed with pharmaceutically acceptable carriers as described supra.

The composition containing the siRNA may be administered to a mammalian subject by a variety of methods described supra. The optimal route of administration is dependent upon the area of the body where suppression of TNFα is most desired. For diseases associated with systemic rises in TNFα, the dosage of siRNA administered can be guided by serum ELISA measurements for levels of this cytokine. In mammalian subjects where systemic intravenous administration is desired, siRNA can be infused via a portable volumetric infusion pump at a rate between about 1-6 mL/hour depending on the volume to be infused as is understood by one of skill in the art. Doses of 0.1 mg/kg/day to about 10 mg/kg/day may be administered for a time period necessary to suppress TNFα expression.

Suppression of the cytokine TNFα is desirable in a variety of immune disorders that include but are not limited to septic shock, rheumatoid arthritis, transplant rejection, scleroderma, immune mediated diabetes, chronic inflammatory bowel syndrome, HIV, cancer, colitis, Crohn's diseaseand inflammation associated with chronic illness. It is desirable to suppress the expression of a molecule on an immune cell such as a cytokine involved in a particular immune related disorder. As such, the invention is applicable to the treatment of a variety of immune disorders associated with the expression of surface markers, enzymes, cytokines, chemokines and transcription factors on an immune cell such as a DC leading to a desired decrease in T cell activity and thus alleviating the immune condition. For the treatment of autoimmune disorders using transformed immune cells of the invention, it is desirable to use the mammalian subjects own cells for transformation and reintroduction into the subject for therapy.

In another embodiment of the invention, the siRNA and/or altered immune cells, in particular DC that exhibits a targeted gene-specific knockout phenotype, can be used in compositions to perfuse cells, tissues and/or organs ex vivo for transplantation. In this aspect, mammalian donor tissues and/or organs are perfused ex vivo with a siRNA composition or transformed immune cell composition of the invention prior to transplantation into a mammalian host. In this manner, the tissue or organ is less susceptible to rejection in the host as T cell activity is suppressed. Methods of tissue/organ perfusion using perfusion machines for example are known to those of skill in the art.

In another embodiment, the invention provides methods for generating tolerogenic dendritic cells (DC) as for example by the suppression of expression of IL-12 on DC using RNAi. Such tolerogenic DC can be used in methods for the treatment of autoimmune disorders where the antigen is known. DC can be isolated from a mammalian subject from bone marrow or peripheral blood and loaded with the autoantigen. These DC are then administered siRNA directed to IL-12 suppression as described supra or in the examples section and then re-infused into the mammalian subject. These DC only generate T regulatory cells and/or Th2 cells specific for the autoantigen. Immunoliposomes specific to DC can be used targeted to a DC-specific surface molecule such as DEC-205, CD11c or CD83, the siRNA may be administered systemically in vivo, in a manner to target DC in homeostatic conditions.

To summarize, the present invention provides novel transformed immune cells which exhibit a targeted gene-specific knockout phenotype in order that such cells can be used therapeutically to modulate immune responses in a mammal via alteration of T cell activity. The present invention provides novel altered DC that do not express one or more genes encoding a surface marker, chemokine, cytokine, enzyme or transcriptional factor that are involved in DC activity, and as such, suppress or stimulate immune system functioning via the modulation of T cell activity.

The present invention also encompasses therapeutic methods for the treatment of a variety of immune disorders with the use of the altered immune cells or with the use of the siRNA. In embodiments of the invention, the immune cells is a DC that is transfected in vitro to produce a desired DC phenotype and then used ex vivo as a perfusion composition for a transplantation tissue or organ or in vivo as administered to a mammalian subject. The invention also encompasses the in vivo use of siRNA directed to selected molecules associated with immune cells in order to alter T cell activity and thus treat a variety of immune disorders.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Generation of Bone Marrow-Derived DC

DC were generated from bone marrow progenitor cells as previously described (22). Briefly, bone marrow cells were flushed from the femurs and tibias of C57BL/6 mice (Jackson Labs, Bar Harbor Me.), washed and cultured in 24-well plates (2×10⁶ cells/ml) in 2 ml of complete medium (RPMI-1640 supplemented with 2 mM L-glutamine, 100 U/ml of penicillin, 100 μg of streptomycin, 50 μM 2-mercaptoethanol, and 10% fetal calf serum (all from Life Technologies, Ontario, Canada) supplemented with recombinant GM-CSF (10 ng/ml; Peprotech, Rocky Hill, N.J.) and recombinant mouse IL-4 (10 ng/ml; Peprotech). All cultures were incubated at 37° C. in 5% humidified CO₂. Non-adherent granulocytes were removed after 48 hrs of culture and fresh medium was added. After 7 days of culture >90% of the cells expressed characteristic DC specific markers as determined by FACS. DC were washed and plated in 24-well plates at a concentration of 2×10⁵ cells per well in 400 μl of serum-free RPMI-1640.

### Example 2

### siRNA Synthesis and Transfection

The siRNA sequences were selected according to the method of Elbashir et al (23). The siRNA sequences specific for IL-12p35 (AACCUGCUGMGGAUGGUGAC), IL-12p40 (MGAUG ACAUCACCUGGACCU), and IFN-γ (MCTGGCAAAAGGATGGTGAC) were synthesized and annealed by the manufacturer (Dharmacon Inc. Lafayette, Co.). siRNA for IFN-γ was used as a control since bone marrow derived DC generated by the conditions described above did not produce IFN-γ after stimulation. Transfection efficiencies were determined using unlabeled and fluorescein labeled siRNA Luciferase GL2 Duplex (Dharmacon Inc). Transfection was carried out as described previously (Elbashir, S. M., 2002. Methods 26:199). Briefly, 3 μl of 20 μM annealed siRNA was incubated with 3 μl of GenePorter (Gene Therapy Systems, San Diego, Calif.) in a volume of 100 μl RPMI-1640 (serum free) at room temperature for 30 min. This was then added to 400 μl of DC cell culture as described above. Mock controls were transfected with 3 μl GenePorter alone. After 4 hrs of incubation an equal volume of RPMI-1640 supplemented with 20% FCS was added to the cells. 24-48 hrs later, transfected DC were washed and used for subsequent experiments.

In the transfection by phagocytosis, bone marrow DC progenitors at day 4 of culture were incubated in a final concentration of 60 pM FL-siRNA-Luc. Cells remained in culture with GM-CSF and IL-4 as described above. At day 8 of culture cells were activated with LPS/TNF-α and incorporated FL-siRNA-Luc was assessed by flow cytometry on day 9.

### Example 3

### DC Activation and MLR

Transfected DC (1×10⁶ cells) were plated in 24 well plates and stimulated with LPS (10 ng/ml, Sigma Aldrich, St Louis, Mo.)+TNFα (10 ng/ml, Peprotech) for 48 hrs, at which point supematants were used for ELISA and RNA was extracted from the cells for RT-PCR. For mixed leukocyte reaction (MLR), T cells were purified from BALB/c splenocytes using nylon wool columns and were used as responders (1×10^{6/}well). siRNA-treated DC (5-40×10³, from C57/BL6 mice) were used as stimulators. 72 hour MLR was performed and the cells were pulsed with 1 μCi [³H]-thymidine for the last 18 hrs. The cultures were harvested on to glass fiber filters (Wallac, Turku, Finland). Radioactivity was counted using a Wallac 1450 Microbeta liquid scintillation counter and the data were analyzed with UltraTerm 3 software.

### Example 4

### Flow Cytometry

Phenotypic analysis of siRNA-treated DC was performed on a FACScan (Becton Dickinson, San Jose, Calif.) and analyzed using CellQuest software (Becton Dickinson). The following FITC conjugated anti-mouse mAbs were used: anti-I-A^{b}, anti-CD11c, anti-CD40, and anti-CD86 (BD PharMingen, San Diego, Calif.). The annexin-V/propidium iodide method of determining apoptosis/necrosis was used as previously described (Min W. P., 2000. J Immunol 164:161). All flow cytometric analyses were performed using appropriate isotype controls (Cedariane Laboratories, Homby ON, Canada).

### Examble 5

### RT-PCR

Total RNA from siRNA-treated DC (10⁶ cells) or from T cells purified from MLR (10⁶ cells) was isolated by TRIzol reagent (Gibco BRL) according to the manufacturer's instructions. First strand CDNA was synthesized using an RNA PCR kit (Gibco BRL) with the supplied oligo d(T)16 primer. One μmol of reverse transcription reaction product was used for the subsequent PCR reaction. The primers used for IL-12p35 and IL-12p40 flanked the sequences targeted by siRNA (IL-12p35, forward primer 5′-GCCAGGTGTCTTAGCCAGTC-3′, reverse primer 5′-GCTCCCTCTTGTTGTGGMG-3′; IL-12p40, forward primer 5′-ATCGTTTTGCTGGTGTCTCC-3′, reverse primer 5′- CTTTGTGGCAGGTGTACTGG-3′). In addition, IL-10, IFN-γ, IL-4 and GAPDH (internal control) primers were used as previously described (Zhu, X., et. al., 1994. Transplantation 58:1104). The PCR conditions were: 94° C. for 1 min, 60° C. for 1 min, and 72° C. for 1 min, and PCR was done for 35 cycles. PCR products were visualized with ethidium bromide on 1.5% agarose gel.

### Example 6

### Enzyme-Linked Immunosorbent Assay (ELISA)

The siRNA-treated DC (10⁵, C57/BL6 origin) were cultured with the allogeneic T cells (1×10⁶) for 48 hrs. The supernatants were harvested and assessed for DC cytokines (IL-12p70, IL-10) and T cell cytokines (IFN-γ, IL-4) by ELISA. Cytokine specific ELISA (Endogen, Rockford, Ill.) was used for detecting cytokine concentrations in culture supematants according to the manufacturer's instructions using a Benchmark Microplate Reader (Bio-Rad Laboratories).

### Example 7

### Immunization of Mice with Peptide-Pulsed DC

Day 7 bone marrow-derived DC were transfection with siRNA-IL12p35, or transfection reagent alone as described above, and pulsed with 10 μg/ml of keyhole limpet hemocyanin (KLH) (Sigma-Aldrich Rockford Ill.) for 24 hrs. DC were then activated with LPS+TNFα for 24hrs, washed extensively and used for subsequent transfer experiments. Antigen-pulsed DC (5×10⁵ cells/mouse) were injected subcutaneously into syngeneic mice. Mice were sacrificed after 10 days and cell suspensions were prepared from the draining lymph nodes. These cells were cultured in 96-well plates at a concentration of 4×10⁵ cells/well in the presence or absence of antigen for 48 hrs at which point culture supernatants were used for analysing cytokine production by ELISA.

For statistical analysis, one-way ANOVA followed by the Newman Keuls Test was used to determine the significance between groups for cytokine production and MLR. Differences with p-values less than 0.05 were considered significant.

Although preferred embodiments have been described herein in detail it is understood by those of skill in the art that using no more than routine experimentation, many equivalents to the specific embodiments of the invention described herein can be made. Such equivalents are intended to be encompassed by the scope of the claims appended hereto.

1. A mammalian immune cell exhibiting a targeted endogenous gene-specific knockout phenotype, said immune cell altering an immune response in a mammal via the modulation of T cell activity.

2. The immune cell of claim 1, wherein said cell comprises a construct that inhibits the expression of said endogenous target gene.

3. The immune cell of claim 2, wherein said construct is selected from the group consisting of siRNA and hybrid DNA/RNA/

4. The immune cell of claim 1, wherein said endogenous gene encodes a surface marker, a chemokine, a cytokine, an enzyme or a transcriptional factor.

5. The immune cell of claim 1, wherein said immune cell is selected from the group consisting of an endothelial cell and an antigen presenting cell.

6. The immune cell of claim 5, wherein said antigen presenting cell is selected from the group consisting of a dendritic cell, a macrophage, a myeloid cell, a B lymphocyte and mixtures thereof.

7. The immune cell of claim 6, wherein said immune cell is a dendritic cell.

8. The immune cell of claim 7, wherein said dendritic cell is activated.

9. The immune cell of of claim 1, wherein said siRNA or hybrid DNA/RNA is provided within a plasmid or vector.

10. The immune cell of claim 9, wherein said plasmid or vector additionally comprises an expressible nucleic acid sequence encoding an antigen.

11. The immune cell of claim 7 or 8, wherein said dendritic cell additionally comprises tumor cell mRNA.

12. The immune cell of claim 4, wherein said surface marker, chemokine, cytokine, enzyme or transcription factor is selected from the group consisting of TNFα, IL-1, IL-1b, IL-2, TNFβ, IL-6, IL-7, IL-8, IL-23, IL-15, IL-18, IL-12, IFNγ, IFNα, lymphotoxin, DEC-25, CD11c, CD40, CD80, CD86, MHCI, MHCII, ICAM-1, TRANCE, CD200, CD200 receptor, CD83, CD2, CD44, CD91, TLR-4, TLR-9, 4-1BBL, nicotinic receptor, GITR-L, OX-40L, CD-CK1, TARC/CCL17, CCL3, CCL4, CXCL9, CXCL10, IKK-β, NK-κB, STAT4, ICSBP/IFN, regulatory factor 8, TRAIL, Inos, arginase, FcgammaRI and II, thrombin, MIP-1α and MIP-1B.

13. The immune cell of claim 12, wherein said cytokine is selected from IL-12 and TNFα.

14. The immune cell of claim 12, wherein said immune cell inhibits T cell activity.

15. The immune cell of claim 4, wherein said surface marker and enzyme are selected from the group consisting of B7-H1, EP2, IL-10 receptor, VEGF-receptor, CD101, PD-L1, PD-L2, HLA-11, DEC-205, CD36 and indoleamone 2,3-dioxygenase.

16. The immune cell of claim 15, wherein said immune cell stimulates T cell activity.

17. The immune cell of claim 14 or 16, wherein said immune cell is administered to a mammalian subject for the treatment of an immune disorder.

18. The immune cell of claim 17, wherein said immune disorder is selected from the group consisting of septic shock, rheumatoid arthritis, transplant rejection, scleroderma, immune medicated diabetes, chronic inflammatory bowel syndrome, HIV, cancer, colitis, Crohn's disease, Goodpasture's syndrome, Multiple Sclerosis, Grave's disease, Hashimoto's thyroditis, Autoimmune pernicious anemia, Autoimmune Addison's disease, Vitiligo, Myasthenia gravis, Scleroderma, Systemic lupus erythematosus, Primary Sjogren's syndrome, Polymyositis, Pemphigus vulgaris, Ankylosing spondylitis, Acute anterior uveitis, Hypoglycemia and inflammation associated with chronic illness.

19. The immune cell of claim 1, wherein said immune cell is provided as a composition comprising a pharmaceutically acceptable carrier.

20. The immune cell of claim 19, wherein said composition additionally comprises an adjuvant and/or antigen.

21. A medicament for the treatment of an immune disorder characterized by inappropriate T cell activity, said medicament comprising a mammalian immune cell that exhibits a targeted gene-specific knockout phenotype, wherein said gene is selected from one or more of a surface marker, a chemokine, a cytokine, an enzyme and a transcriptional factor.

22. A medicament for the treatment of an immune disorder characterized by inappropriate T cell activity, said medicament comprising a siRNA possessing specific homology to part or the entire exon region of a gene encoding a surface marker, a chemokine, a cytokine, an enzyme or a transcriptional factor or an antigen presenting cell (APC).

23. The medicament of claim 20 or 21, wherein said gene is selected from the group consisting of TNFα, IL-1, IL-1b, IL-2, TNFβ, IL-6, IL-7, IL-8, IL-23, IL-15, IL-18, IL-12, IFNα, lymphotoxin, DEC-25, CD11c, CD40, CD80, CD86, MHCI, MHCII, ICAM-1, TRANCE, CD200, CD200 receptor, CD83, CD2, CD44, CD91, TLR4, TLR-9, 4-1BBL, nicotinic receptor, GITR-L, OX40L, CD-CK1, TARC/CCL17, CCL3, CCL4, CXCL9, CXCL10, IKK-β, NF-κB, STAT4, ICSBP/IFN, regulatory factor 8, TRAIL, Inos, arginase, FcgammaRI and II, thrombin, MIP-1α and MIP-1B.

24. The medicament of claim 22, wherein said T cell activity is inhibited.

25. The medicament of claim 20 or 21, wherein said gene is selected from the group consisting of B7-H1, EP2, IL-10 receptor, VEGF-receptor, CD101, PD-L1, PD-L2, HLA-11, DEC-205, CD36 and indoleamine 2,3-dioxygenase.

26. The medicament of claim 25, wherein said T cell activity is stimulated.

27. The medicament of claim 23 or 24, wherein said immune disorder is selected from the group consisting of septic shock, rheumatoid arthritis, transplant rejection, scleroderma, immune mediated diabetes, chronic inflammatory bowel syndrome, HIV, cancer, colitis, Crohn's disease, Goodpasture's syndrome, Multiple Sclerosis, Grave's disease, Hashimoto's thyroditis, Autoimmune pernicious anemia, Autoimmune Addison's disease, Vitiligo, Myasthenia gravis, Scleroderma, Systemic lupus erythermaosus, Primary Sjogren's syndrome, Polymyositis, Pemphigus vulgaris, Ankylosing spondylitis, Acute anterior uveitis, Hypoglycemia and inflammation associated with chronic illness.

28. The medicament of claims 21 to 22 wherein said immune cell is selected from an endothelial cell and an antigen presenting cell (APC).

29. The medicament of claim 28, wherein said antigen presenting cell is selected from the group consisting of a dendritic cell, a macrophage, a myeloid cell, a B lymphocyte and mixtures thereof.

30. The medicament of claim 29, wherein said immune cell is a dendritic cell.

31. The medicament of claim 30, wherein said dendritic cell is activated.

32. A composition for the treatment of an immune disorder, said composition comprising at least one of:
(a) a construct that inhibits the expression of an endogenous target gene encoding a surface marker, a chemokine, a cytokine, an enzyme or a transcriptional factor in an immune cell such that said immune cells alters T cell activity; and
(b) an immune cell wherein said immune cell comprises at least one construct that inhibits the expression of an endogenous target gene encoding a surface marker, a chemokine, a cytokine, an enzyme or a transcriptional factor; and
(c) a pharmaceutically acceptable carrier,
wherein said composition alters T cell activity leading to an altered immune response.

33. The composition of claim 32, wherein said construct is selected from the group consisting of siRNA and hybrid DNA/RNA.

34. The composition of claim 32, wherein said immune cell is selected from the group consisting of an endothelial cell and an antigen presenting cell.

35. The composition of claim 34, wherein said antigen presenting cell is selected from the group consisting of a dendritic cell, a macrophage, a myeloid cell, a B lymphocyte and mixtures thereof.

36. The composition of claim 35, wherein said immune cell is a dendritic cell.

37. The composition of claim 36, wherein said dendritic cell is activated.

38. The composition of claim 33, wherein said siRNA or hybrid DNA/RNA is provided within a plasmid or vector.

39. The composition of claim 38, wherein said plasmid or vector additionally comprises an expressible nucleic acid sequence encoding an antigen.

40. The composition of claim 35, wherein said dendritic cell additionally comprises tumor cell mRNA.

41. The composition of claim 32, wherein said surface marker, chemokine, cytokine, enzyme or transcription factor is selected from the group consisting of TNFα, IL-1, IL-1b, IL-2, TNFβ, IL-6, IL-7, IL-8, IL-23, IL-15, IL18, IL-12, IFNγ, IFNα, lymphotoxin, DEC-25, CD11c, CD40, CD80, CD86, MHCI, MHCII, ICAM-1, TRANCE, CD200, CD200 receptor, CD83, CD2, CD44, CD91, TLR4, TLR-9, 4-1BBL, nicotinic receptor, GITR-L, OX-40L, CD-CK1, TARC/CCL17, CCL3, CCL4, CXCL9, CXCL10, IKK-β, NF-κB, STAT4, ICSBP/IFN, regulatory factor 8, TRAIL, Inos, arginase, FcgammaRI and II, thrombin, MIP-1α and MIP-1B.

42. The composition of claim 41, wherein said cytokine is selected from IL-12 and TNFα.

43. The composition of claim 32, wherein said surface marker and enzyme are selected from the group consisting of B7-H1, EP2, IL-10 receptor, VEGF-receptor, CD101, PD-L1, PD-L2, HLA-11, DEC-205, CD36 and indoleamine 2,3-dioxygenase.

44. The composition of claim 32, wherein said immune disorder is selected from the group consisting of septic shock, rheumatoid arthritis, transplant rejection, scleroderma, immune mediated diabetes, chronic inflammatory bowel syndrome, HIV, cancer, colitis, Crohn's disease, Goodpasture's syndrome, Multiple Sclerosis, Grave's disease, Hashimoto's thyroditis, Autoimmune pernicious anemia, Autoimmune Addison's disease, Vitiligo, Myasthenia gravis, Scleroderma, Systemic lupus erythermaosus, Primary Sjogren's syndrome, Polymyositis, Pemphigus vulgaris, Ankylosing spondylitis, Acute anterior uveitis, Hypoglycemia and inflammation associated with chronic illness.

45. The composition of claim 32, wherein said composition is used to perfuse tissues and/or organs ex vivo.

46. A method for inhibiting the T cell activating ability of a DC, the method comprising transforming said DC with a constructcapable of inhibiting the expression of an endogenous target gene encoding a surface marker, a chemokine, a cytokine, an enzyme or a transcriptional factor.

47. A method of decreasing the immunogenicity and rejection potential of an organ for transplantation, said method comprising perfusing said organ with a composition that suppresses T cell activity, said composition comprising at least one construct that inhibits the expression of an endogenous target gene encoding a surface marker, a chemokine, a cytokine, an enzyme or a transcriptional factor and a pharmaceutically acceptable carrier.

48. The method of claim 46 or 47, wherein said construct is selected from siRNA and hybrid DNA/RNA.

49. The method of claim 48, wherein said siRNA is provided within an antigen presenting immune cell.

50. A method for making a immune cell that alters that activity of T cell in vivo, said method comprising;
transforming immune cells in vitro with at least one construct that inhibits the expression of an endogenous target gene encoding a surface marker, a chemokine, a cytokine, an enzyme or a transcriptional factor.

51. A method for the treatment of autoimmune disorders and transplantation rejection in a mammalian subject, said method comprising administering a therapeutically effective amount of a composition to said subject, said composition comprising DC that contain at least one construct that inhibits the expression of an endogenous target gene encoding a surface marker, a chemokine, a cytokine, an enzyme or atranscriptional factor, wherein said DC suppresses T cell activity.

52. The method of claim 50 or 51, wherein said construct is selected from siRNA and hybridDNA/RNA.

53. A method for the treatment of autoimmune disorders and transplantation rejection in a mammalian subject, said method comprising administering a therapeutical effective amount of a composition to said subject, said composition comprising an siRNA targeted to inhibit expression of an endogenous target gene in an antigen presenting cell, said gene encoding a surface marker, a chemokine, a cytokine, an enzyme or a transcriptional factor, wherein said siRNA suppresses T cell activity.

54. The method of claims 51, 52 or 53, wherein said autoimmune disorder is selected from the group consisting of septic shock, rheumatoid arthritis, transplant rejection, scleroderma, immune mediated diabetes, chronic inflammatory bowel syndrome, HIV, cancer, colitis, Crohn's disease, Goodpasture's syndrome, Multiple Sclerosis, Grave's disease, Hashimoto's thyroditis, Autoimmune pernicious anemia, Autoimmune Addison's disease, Vitiligo, Myasthenia gravis, Scleroderma, Systemic lupus erythematosus, Primary Sjogren's syndrome, Polymyositis, Pemphigus vulgaris, Ankylosing spondylitis, Acute anterior uveitis, Hypoglycemia and inflammation associated with chronic illness.
